(19) United States
(12) Patent Application Publication
(10) Pub. No.: US 2006/0127459 A1
(43) Pub. Date: Jun. 15, 2006

(54) Urogenital infection inhibition

(76) Inventors: Lei Huang, Duluth, GA (US); Shu-Ping Yang, Alpharetta, GA (US); Yanbin Huang, Foster City, CA (US)

Correspondence Address: KIMBERLY-CLARK WORLDWIDE, INC., 401 NORTH LAKE STREET, NEENAH, WI, 54956, US

(21) Appl. No.: 11/012,758

(22) Filed: Dec. 15, 2004

(51) Int. Cl. A61K 31/765; A61K 9/70

(52) U.S. Cl. 424/443; 424/78.38

(57) ABSTRACT

There is provided compositions and methods to inhibit the adherence of pathogenic microorganisms to cells. The composition includes water soluble PEO/PEG polymers which may be hydrophilic or amphiphilic block copolymers. One example of a suitable composition is polyethylene glycol or polyethylene oxide with a chemical formula of H—(OCH2CH2)n-OH. Another example of a suitable composition is amphiphilic block copolymer with a chemical formula PEOm-Xp-PEOn, where PEO is —(OCH2CH2)n-O and X is PPO(—(OCH2CH2CH2)m-), or PLGA (poly(lactic-co-glycolic acid), or PMMA (polymethyl methacrylate), or PBO (polybutylene oxide). The pathogenic microorganism may be a bacterial, fungal, viral or trichomonial pathogen and the composition prevents adherence of the pathogenic microorganism in, for example, a urogenital tract of a mammal and aids in reducing malodor generation. The composition may be applied to vaginal health products like, for example, moisturizer, gel, jelly, cream, insert (tablet), ointment, foam. The composition may also be applied to a feminine hygiene product, like, for example, a tampon, feminine pad, feminine wipe or panty liners.

### BACKGROUND OF THE INVENTION

The invention relates to the prevention of the attachment of pathogens to vaginal tissues. Those pathogens in the vagina frequently are a major cause of infection and malodor generation.

The vagina is fairly resistant to infection due to its marked acidity, balanced ecosystem and thick protective epithelium. However, numerous insults can affect the vaginal defense system and lead to increased susceptibility to vaginal infection. For example, low estrogen levels in menopausal and hypogonadal women can affect the thickness of the vaginal epithelium. Antibiotics can alter the microbiology of the vagina. Semen during intercourse and blood during menstruation can increase vaginal pH. Stress, fatigue, chronic diseases such as diabetics and human immunodeficiency disease (HIV) affect not only the immune system but also the pH of the vagina. These factors can breakdown the balanced microenvironment in the vagina and increase the risk of vaginal infection by a variety of organisms. During the menstrual period women may also have increased risk for pathogenic microorganism activity due to the presence of menstrual fluid. After the menstrual cycle, many women complain about the discomfort of symptoms like vaginal dryness and unpleasant odors. These symptoms are most probably caused by diluted “lubricants”—secretions from the vaginal wall, and the increase in pathogenic microorganism activity due to the exposure of menstrual fluid to the vaginal canal.

Candida, which is a type of yeast or fungus, is normally found in the body along with bacteria such as E. coli. When the human body is in balance it usually causes no problems. When the internal environment is out of balance from stress or fatigue, those microorganisms (yeast and bacteria) can grow disproportionately, cause infection, and generate malodor. Colonization of the vagina by pathogenic bacteria such as Escherichia coli is a significant step in ascending urinary tract infections (UTIs), which affect about 10-20% of women at some time in their life and which cost about $US 5 billion per year in healthcare costs. Estrogen-depleted post-menopausal women are the highest risk group for acquiring urinary tract infections due to the thinning of the vaginal mucosa and the increased pH of the vaginal environment. Recurrences of both vaginal and urinary tract infections are frequent following the initial episode. Hence, prevention of the initial infection is important for avoiding repeated urogenital infections.

Without treatment, vaginal infections can increase the risk of sexually transmitted diseases and induce complications such as urinary tract infections, pelvic inflammatory diseases and pre-term births. Currently, the control of such infections relies heavily on antibiotics. However, extensive use of antibiotics for prevention of infection can be detrimental, not only because of the increased risk of generating antibiotic-resistant microorganisms, but also because indiscriminate killing of beneficial bacteria in the urogenital tract can leave it susceptible to infection by other pathogens. Hence, there is increasing concern that compositions containing antibiotics should not be used routinely for treating urogenital infections.

Thus, a need exists for compositions and methods for treating and, especially for preventing, urogenital infections, without the use of antibiotics or harsh chemicals that can upset the natural balance within the urogenital tract. A need further exists for compositions and methods of application in feminine health and hygiene products for preventing pathogen associated malodor generation.

### SUMMARY OF THE INVENTION

The present invention is directed to compositions and methods for treating and preventingpreventing infections within the urogenital tract, for example, within the vagina and urinary tract. It is also directed to compositions for use in feminine health and hygiene products for preventing pathogen associated malodor generation by reducing/preventing pathogen attachment to vaginal tissues.

Disclosed are compositions and methods of treating and preventingpreventing infection by the “anti-adhesive” approach; where compounds are used to inhibit the attachment between pathogens and cell/tissues, the first and critical step for pathogens to establish an infection.

The invention is directed to using water soluble water soluble hydrophilic PEO/PEG polymers for infection control and pathogen associated malodor prevention.

Pathogens that can be treated by the compositions, methods and articles of the invention include bacteria, yeast, fungi, viruses, trichomonia and other parasites. An effective amount of the compounds of the invention can vary, but in some embodiments, the effective amount can range from about 1 and 50 weight percent. In other embodiments the compound may be applied in a more pure form or applied as a solution and allowed to dry on a substrate.

### BRIEF DESCRIPTION OF THE DRAWINGS

FIG. 1 is a drawing of various types of conformation of PEO containing amphiphilic block copolymers.

FIG. 2 is a drawing of a tampon.

FIG. 3-8 show in bar-graph form the growth of bacterial colony forming units in various media and on various substrates.

### DETAILED DESCRIPTION

Urogenital infections often result from an imbalance in the types of microflora that occupy the urogenital tract, for example, a vagina with an overabundance of one type of bacteria is subject to infection by that type of bacteria as well as being more susceptible to infection by other types of bacteria, fungi, viruses and other parasites. The initial and critical step in the pathogenesis of urogenital infections involves adherence by the pathogen to the epithelium of the urogenital tract (e.g., the vagina). Provided herein are compositions and methods to inhibit adherence of pathogens to the epithelium of the urogenital tract through the use of inexpensive, readily available active compounds. The compositions and methods avoid strong chemicals and unnatural substances whose effects on the health and reproduction of the user are unknown. Use of these compositions and methods improves the microenvironment in the urogenital tract, reduces the number of pathogens occupying the urogenital tract and prevents growth and attachment of an overabundance of pathogens to urogenital tissues.

One mechanism by which pathogens adhere to host tissue cells is through protein-sugar interactions. The adherence of Candida albicans onto female genital tract cells, for example, involves binding between proteins on the fungal cell surface and fructose-containing glycosides on the epithelial surface. The adherence of uropathogenic E. coli to the urinary tract involves binding of bacterial PapG Adhesin and FimH Adhesion proteins to epithelial cell surface sugar residues (for example, Gal(α1,4)Gal from glycolipids and mannose-residues on the mammalian cell surface). The adherence of Chlamydia trachomatis to the genital tract involves binding between a Chlamydia surface polysaccharide (containing 7-9 mannose residues) and mannose-binding proteins on the host cell surface. Adherence of Mycoplasma bovis to the genital and urinary tract involves binding between bacterial surface proteins and sialic acid residues on the host cell surface. Urinary tract infection by Staphylococcus saprophyticus involves the binding between bacterial surface lectins and the GalNac residues on the host cell surface.

Another mechanism of bacterial adherence to host tissues is through non-specific binding by, for example, hydrogen-bonding and divalent-cation-mediating binding between the glycocalyx layers of bacteria and host cells. A good pathogen adhesion inhibiting system should prevent both the specific and the non-specific binding of pathogens to vaginal and urinary tract cell surfaces.

Adherence by many types of pathogens can be inhibited by the compositions and methods disclosed herein. Such pathogens can be fungi, bacteria, viruses, trichomonia or other parasites. Adherence of urogenital Candida, bacteria, genital herpes, chlamydia, trichomonia, gonorrhea and human papilloma virus may also be inhibited.

Genital candidiasis, generally known as yeast infection, is the infection of the genital tract caused by Candida albicans. Women suffering from yeast infection usually develop vulval irritation, itching and vaginal discharge, the vaginal wall is covered with a white cheesy material, and the vulva is reddish and swollen.

Bacterial infections by bacteria such as Escherichia coli, Gardnerella vaginalis, Mycoplasma bovis, Mycoplasma hominus, Neisseria gonorrhoeae, Staphylococcus saprophyticus, may also be inhibited by the compositions and methods disclosed herein.

Viral infections by organisms such as human papilloma virus, herpes simplex virus type 2 and herpes simplex virus type 1 may also be inhibited by the compositions and methods disclosed herein.

Chlamydial infections are sexually transmitted nongonococcal infections caused by Chlamydia trachomatis. These infections include nongonococcal urethritis, mucopurulent cervicitis and nonspecific genital infections. Typically, the affected individual suffers from vaginal discharge, dysuria and cervicitis with yellow, mucopurulent secretion. Infections by Chlamydia trachomatis may also be inhibited by the compositions and methods disclosed herein.

Trichomoniasis is caused by a flagellate anaerobic protozoan Trichomonas vaginalis. The trichomoniasis is accompanied by a copious, greenish-yellow, frothy vaginal discharge associated with irritation, itching and soreness of the vulva and thighs. The vaginal walls and cervix surface show punctuate red spots. Infections by Trichomonas vaginalis may also be inhibited by the compositions and methods disclosed herein.

Polyethylene oxide (PEO) is a hydrophilic crystalline thermoplastic polymer with the chemical formula of —(OCH2CH2)n-O. It is biocompatible and also is very “protein-resistant”. In the biomedical field, surfaces treated with hydrophilic polyethylene oxide (PEO) and polyethylene glycol (PEG) H—(OCH2CH2)n-OH have been shown to have protein adsorption resistant and antifouling properties. The PEO surfaces have been prepared, however, by physical adsorption of PEO onto substrates and the resulting non-water soluble PEO modified surfaces have been shown to increase wettability and to render surfaces resistant to protein adsorption. The process of surface PEO modification is expensive, and may involve grafting (chemical immobilization), cross-linking and plasma deposition. Most of the resulting “protein-resistant” or “non-fouling” surfaces are widely used in markets where such high costs may be borne, such as in biomedical devices.

In contrast, the inventors have found that water soluble PEO/PEG polymers have pathogen adhesion inhibition properties. These polymers may reduce the adhesion of pathogens on epithelial cells by at least 10 percent as compared to a control (without the polymers), particularly at least 25 percent and most particularly at least 50 percent. The water soluble PEO/PEG polymers may be hydrophilic or amphiphilic.

The inventors further have found that water soluble PEO/PEG polymers can be used in consumable vaginal health products, like tampons, feminine pads, feminine wipes or panty liners or vaginal moisturizers, without the use of expensive covalent binding processes. Water soluble PEO polymer/copolymers can be incorporated into vaginal health products by traditional nonwoven processes, like spray coating or dip coating (also known as dip and squeeze), ink-jet printing and other methods known in the art. The outer cover of a tampon, feminine pad or wiper, for example, may be coated with the water soluble composition.

It is expected that the water soluble PEO based polymer/copolymer coating will be gradually dissolved and released into the vaginal canal environment during tampon wearing and/or moisturizer use. The released polymer/copolymer not only can function as a moisturizer to ease vaginal dryness, but can also lubricate the vaginal wall and reduce or prevent pathogenic microorganism's adhesion and thus control odor by reducing or preventing vaginal infection.

PEO-based polymers that are hydrophilic include those with the general chemical formula —(OCH2CH2)n-O or H—(OCH2CH2)n-OH, where the number-average molecular weight is 6,000 or higher and include 20 k PEG and 900 k PEO available from Sigma-Aldrich Chemical Company of Milwaukee, Wis.

PEO-based polymers with amphiphilic block copolymers also have inhibitory properties for the binding between pathogens and epithelial cells. The general chemical formula for amphiphilic block copolymers is: PEOm-Xp-PEOn, where PEO is —(OCH2CH2)n-O and X is PPO(—(OCH2CH2CH2)m-), or PLGA (poly(lactic-co-glycolic acid), or PMMA (polymethyl methacrylate), or PBO (polybutylene oxide). The values of n, m, and p can be same or different. The amphiphilic block copolymer may use, for example, poly(ethylene oxide) (PEO) as the hydrophilic block, and may use poly(propylene oxide) (PPO), or poly(lactic-co-glycolic acid) (PLGA), or PMMA (polymethyl methacrylate), or PBO (polybutylene oxide) as the hydrophobic block, where the number-average molecular weight is 6,000 or higher.

Suitable examples of water soluble PEO polymers include the PLURONIC® and PLURONIC® R block copolymer series, TETRONIC® block copolymer and TETRONIC®) R block copolymer series commercially available from BASF. It is hypothesized here, though the inventors do not wish to be bound by this hypothesis, that the hydrophobic block binds with the cell surface, while the hydrophilic blocks dangle around and form an effective barrier for pathogen to attach to cell surface.

Amphiphilic block copolymers have both hydrophobic and hydrophilic parts. An example of a particular amphiphilic block copolymer found to be useful is PLURONIC® F127 (Poloxamer 407NF from BASF).

In one aspect, the general chemical formula for amphiphilic block copolymers is:

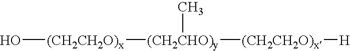

A suitable example of the amphiphilic block copolymers with the above structure are the PLURONIC®) series commercially available from BASF. The PLURONIC® series are block copolymers of propylene oxide and ethylene oxide. The propylene oxide (PPO) block is sandwiched between two ethylene oxide (PEO) blocks with different total length and EO/PO compositions.

In another aspect, the general chemical formula for amphiphilic block copolymers is:

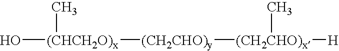

A suitable example of the amphiphilic block copolymers with the above structure are the PLURONIC® R series commercially available from BASF. The PLURONIC® series are block copolymers of propylene oxide and ethylene oxide. The ethylene oxide (PEO) block is sandwiched between two propylene oxide (PPO) blocks with different total length and EO/PO compositions.

In another aspect, the general chemical formula for amphiphilic block copolymers is:

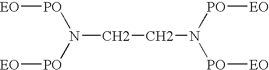

A suitable example of the amphiphilic block copolymers with the above structure are the TETRONIC® series commercially available from BASF. The TETRONIC® series are tetra-functional block copolymers consisting of block copolymers of PEO-PPO-PEO with different total length and EO/PO compositions.

In another aspect, the general chemical formula for amphiphilic block copolymers is:

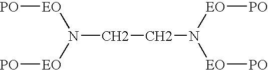

A suitable example of the amphiphilic block copolymers with the above structure can be the TETRONIC® R series commercially available from BASF. The TETRONIC® R series are also tetra-functional block copolymers consisting of block copolymers of PEO-PPO-PEO with different total length and EO/PO compositions. They have different hydrophobic and hydrophilic blocks when compared with the TETRONIC® series.

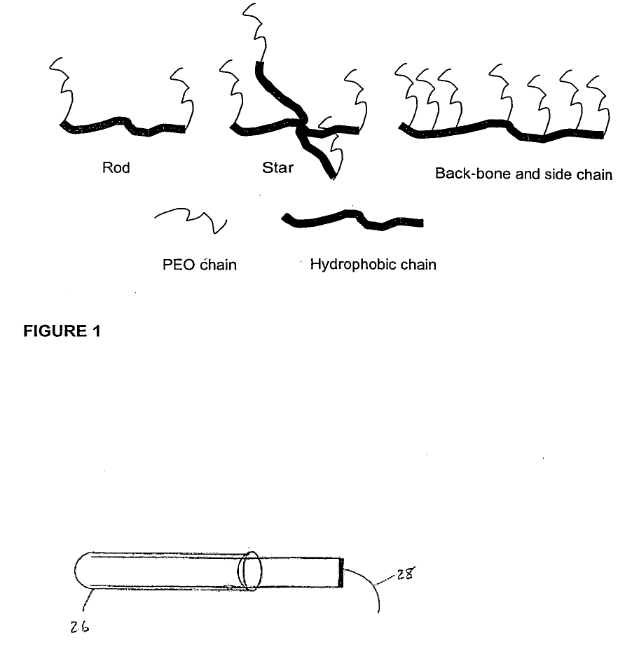

Different conformation types of amphiphilic block copolymers for infection control and pathogen associated malodor prevention may also be used. Suitable examples of amphiphilic block copolymers' conformations are shown in FIG. 1.

In some embodiments the suitable concentrations of the polymers are between about 1 and 50 weight percent, more particularly between 2 and 20 weight percent and still more particularly between 5 and 10 weight percent with the balance being a buffer. In other embodiments the polymer may be used in a more pure form (up to 100 weight percent) or applied as a solution and allowed to dry on a substrate, also producing a near pure form of polymer. The polymer may be applied, for example, in a solution by the dip and squeeze method followed by oven drying at a temperature and for a time sufficient to dry the solution on the substrate. Alternative methods include spray coating, ink-jet printing and other techniques known in the art. The polymer may be an ingredient for vaginal health products like, for example, moisturizer, gel, jelly, cream, insert (tablet), ointment, foam. The polymer may also be applied to a feminine hygiene product, like, for example, a tampon, pad, or pant liners, which can reduce/prevent pathogenic microorganism's adhesion and prevent pathogen associated malodor.

Suitable buffers for use withwith the PEO and PEG polymers and copolymers are water soluble, non-toxic, do not interfere with biological processes, and do not interfere with biological membranes (penetration, solubilisation, adsorption on surface etc.) Examples of suitable buffers for testing the PEO and PEG polymers and copolymers used herein include phosphate buffered saline (PBS) solution (pH of 7.2), tris-buffered saline (TBS) solution (pH of 8.2) or 2-(N-morpholino) ethane sulfonic acid (MES) (pH of 5.3).

PEO based hydrophilic polymers (like PEG) and amphiphilic block copolymers (like PLURONIC® F127 and TETRONIC® T150 R1) having the proper molecular weight are water soluble and can be used in consumable vaginal health products, like tampons, feminine hygiene pads, wipes or in vaginal moisturizers, without the use of expensive covalent binding processes.

A drawing of a typical tampon is shown in FIG. 2. Tampons have the general structure as described in U.S. Pat. Nos. 3,520,302 and 3,683,912 which are incorporated herein in their entirety by reference thereto for all purposes. The generally elongate shape of a tampon typically has an absorbent body 26 and a withdrawal string 28. The absorbent body 26 may be wrapped by an outer cover (not shown). Additional structural features may also be present.

A series of experiments was conducted to investigate different molecular weights of water soluble polymers' having potential to be used for vaginal health and pathogen associated malodor prevention.

The results were quite positive and are reported below.

Experiment 1: Testing of 20 k PEG and 900 k PEO polymers for their ability to inhibit C. albicans cell adherence using monolayer vaginal epithelium cell model was performed using the following materials and method.

PEG 20 k (from Sigma-Aldrich, Cat #:81300)

PEO 900 k (from Sigma-Aldrich, Cat #:18,945-6)

Hyaluronic acid (HLA) potassium salt (from Sigma-Aldrich Cat# No. H-1751) (positive control) which has been shown to inhibit pathogen adherence to epithelium cells as shown in commonly assigned U.S. patent application Ser. No. 10/401,522.

C. albicans: ATCC 10231

A431 epithelial cells, ATCC CRL-1555

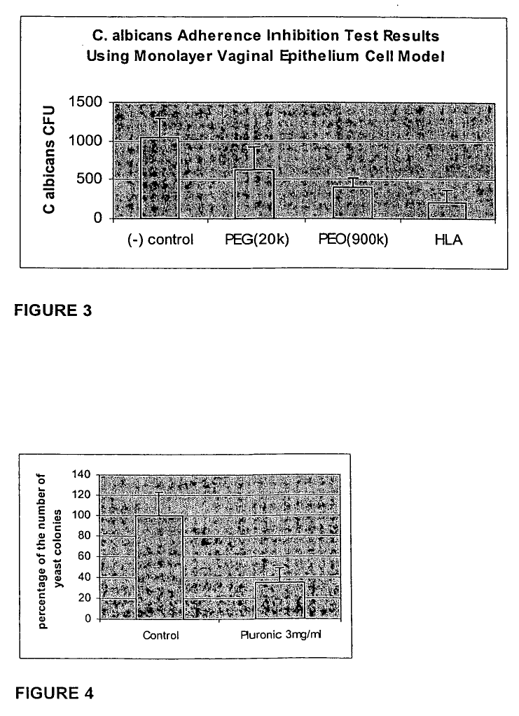

5 mg/ml polymer solutions were prepared using sterilized 1×PBS solution. A monolayer of A431 epithelial cells, ATCC CRL-1555, was grown on a 24-well tissue culture plate until confluent. 1.0 ml of PBS control or polymer solution samples was added onto each epithelial tissue and incubated at 37° C. for 30 minutes. We then removed 0.5 ml of the liquid solution and added 0.5 ml of yeast TSB (Trypticase™ Soy Broth) suspension (at a concentration of 1×10⁶ cfu/ml). After two hours of incubation, the supernatant was removed from the wells and the wells were rinsed thoroughly three times with PBS to remove all non-bound yeast. The bound bacteria were re-grown in 5 ml TSB and the whole system was incubated at 37° C. with shaking for 4 hours. The TSB was plated on Sabouraud's dextrose agar plates after 1×, 0.1×, 0.01× serial dilutions. The plates were incubated overnight at 35° C. and the number of colonies on each plate was counted. The results are also shown graphically in FIG. 3 where the Y axis is the C. albicans colony forming units (CFU) measured on the epithelial cells and the X axis is the type polymer in the order: control, 20 k PEG, 900 k PEO, HLA. The bar in FIG. 3 represents mean±SEM and N=4/group. The data were statistically analyzed by t-Test and the results are considered significant (p<0.05).

The results illustrated that both 20 k PEG and 900 k PEO hydrophilic polymers inhibited adherence of Candida albicans to mammalian epithelial cells. Candida albicans is the most prevalent pathogen that causes vaginitis.

Experiment 2: This experiment used PLURONIC® F127 as the example amphiphilic block copolymer and C. albicans as the example pathogen to demonstrate the efficacy of amphiphilic block copolymer. The following procedure was used.
1. Cultured Candida albicans (yeast), ATCC 10231, in trypticase soy broth (TSB).
2. Cultured a monolayer of A431 cells, ATCC CRL-1555 on cover slips in a 24-well tissue culture plate until confluent.
3. Dissolve PLURONIC® (PLURONIC® 127 from BASF) in PBS solution to get 3 mg/ml solution.
4. Added 1.0 ml of PBS or PLURONIC®) solutions into each well with confluent A431 cells.
5. Incubated for 30 minutes at 37° C.
6. Removed 0.5 ml of the solution.
7. Added 0.5 ml of the yeast TSB suspension (at a concentration of 5×10⁵ cfu/ml).
8. After two hours of incubation, removed the supernatant from the wells and the wells were rinsed thoroughly with PBS to remove all non-bound yeast.
9. Transferred the cover slips with bound yeast into tubes and re-grew in 5 ml TSB. Incubated the whole system at 37° C. with shaking for 3 hours.
10. Plated the yeast/TSB system on Sabouraud's dextrose agar plates after 1×, 0.1×, 0.01× serial dilutions. Incubated the plates overnight at 35° C. Counted the number of colonies on each plate and calculated back to the number of yeast attached to the cells in each well.
11. Statistics were done with the t-Test. P<0.05 was assigned as significant.

As shown in FIG. 4, the presence of PLURONIC® F127 greatly inhibits the attachment of C. albicans on epithelial cells. In FIG. 4 the Y axis is the relative numbers of yeast colonies from 0 to 140. On the X-axis is a control and 3 mg/ml PLURONIC® sample.

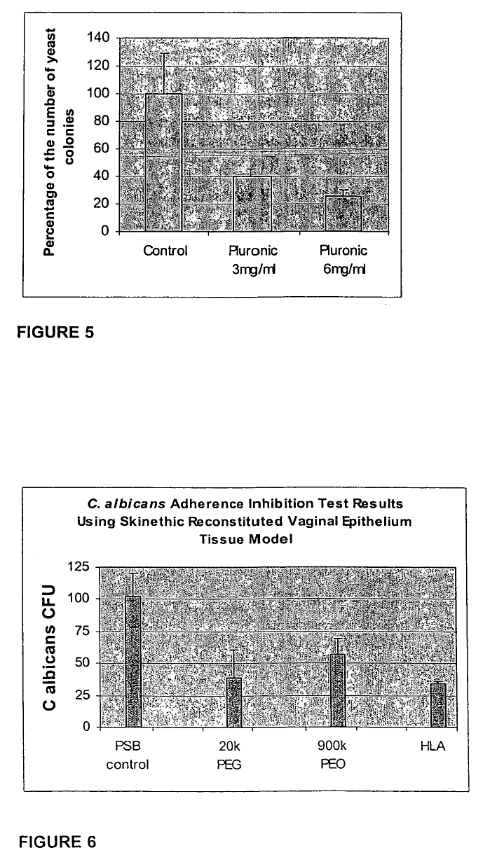

The inhibitory effect of PLURONIC® on C. albicans adherence to epithelial cells was repeated with PLURONIC® solution at concentrations at 3 mg/ml and 6 mg/ml. The result was confirmed that PLURONIC®) solution at both concentrations could significantly inhibit C. albicans adherence on human epithelial cells. FIG. 5 has bars representing both 3 mg/ml and 6 mg/ml PLURONIC® samples. In FIG. 5 the Y axis is the relative numbers of yeast colonies from 0 to 140. On the X-axis is a control, 3 mg/ml PLURONIC® sample and 6 mg/ml PLURONIC® sample.

Experiment 3: Testing 20 k PEG and 900 k PEO for their ability to inhibit C. albicans cell adherence using commercial SKINETHIC® reconstituted vaginal epithelium tissue, available from SkinEthic Laboratories (45, rue Saint-Philippe, 06000 Nice, France).

5 mg/ml 20 k PEG and 900 k PEO polymer solutions were prepared using sterilized 1×PBS solution.

A reconstituted vaginal epithelial tissue model from SKINETHIC® was used, which consists of airlifted, living, multi-layered epithelial tissue produced in polycarbonate inserts in a serum-free and chemically defined medium, featuring normal ultra-structure and functionality equivalent to the epithelia of humans in vivo.

Prior to testing, each 0.5 cm² SKINETHIC® human epithelial tissue was moved into a 24-well plate with 0.5 ml fresh SKINETHIC®) Maintenance (at room temperature) and added 0.5 ml of PBS or polymer solution sample on top of the epithelial tissue, and incubated for 30 minutes at 37° C.

Then 0.5 ml of the yeast TSB suspension (at a concentration of 1×10⁶ cfu/ml) was added. After about two hours of incubation, the liquid was removed. The wells were rinsed thoroughly with PBS to remove all non-bound yeast for three times.

The tissue was cut with a sterilized scalpel and rinsed by PBS again. Then the bound bacteria were re-grown in 5 ml TSB and the whole system was incubated at 37° C. with shaking for 4 hours. The TSB was plated on Sabouraud's dextrose agar plates after 1×, 0.1×, and 0.01× serial dilutions. The plates were incubated overnight at 35° C. The number of colonies on each plate was counted and calculated back to the number of yeast attached to the cells in each well.

FIG. 6 shows the results of adherence testing where the Y axis is the C. albicans colony forming units (CFU) measured on the epithelial cells with a scale of 0 to 125 with increments each 25 units, and the X axis is the type polymer in the order: control, 20 k PEG, 900 k PEO, Hyaluronic acid (HLA).

Both 20 k PEG and 900 k PEO showed significant results in preventing yeast cell adherence using SKINETHIC® reconstituted vaginal epithelium tissue model.

Experiment 4: Effect of Polymers on Yeast C. albicans Growth

This Example illustrates that the decreased adherence of yeast C. albicans to A431 cells and SKINETHIC® reconstituted vaginal epithelium tissue model was not due to an inhibitory effect by polymers on yeast C. albicans growth.

Materials and Methods: To test whether PEO based polymer and copolymers could influence cellular growth, C. albicans, ATCC 10231 cells were cultured overnight in TSB. 5 mg/ml 20 k PEG, 900 k PEO and PLURONIC® F127 polymer solutions were prepared using sterilized 1×PBS solution. Then 0.1 ml of the diluted yeast C. albicans TSB suspension (at a concentration of 1×10⁶ cfu/ml) was added into 0.9 ml polymer PBS solutions respectively along with PBS control solution. The test and control cells were incubated at 37° C. with shaking and the sample solutions were withdraw at 5 minutes and 2 hours after addition of the polymer PBS solutions or PBS. The withdraw sample solution was plated on Sabouraud's dextrose agar plates after 1×, 0.1×, and 0.01× serial dilutions. The plates were incubated overnight at 35° C. The number of colonies on each plate was counted and calculated back to the number of live yeast in each test sample solution.

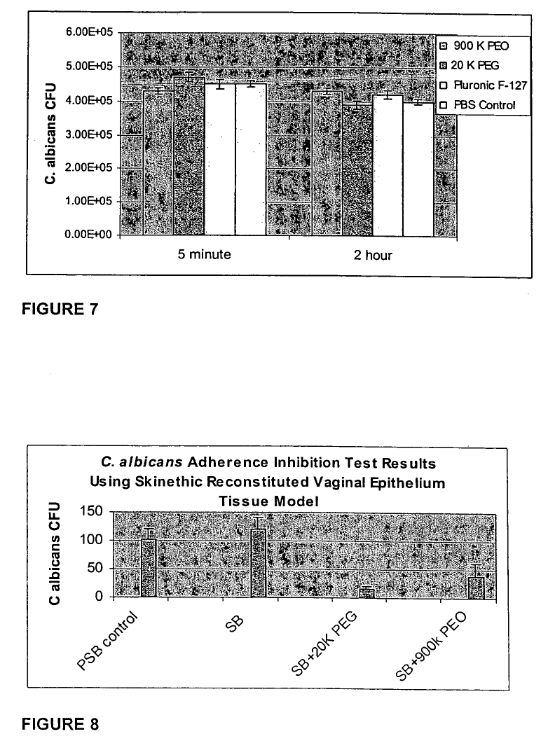

The results of the effect of PEO and PEG based polymer and copolymers on C. albicans growth are shown in FIG. 7. As illustrated in FIG. 7, the PEO and PEG based polymer and copolymer solutions did not have any effect on yeast growth when compared with PBS control under the experimental conditions. In the graph of FIG. 7 the Y axis is the C. albicans colony forming units (CFU) measured on the epithelial cells with a scale of 0 to 6×10⁵ with increments each 10⁵ units, and the X axis is the type polymer in the order: 900 k PEO, 20 k PEG, PLURONIC® F127 and control. These data indicate that the inhibition of yeast C. albicans attachment to A431 cells and SKINETHIC® reconstituted vaginal epithelium tissue model shown in Example 1-3 was not due to inhibition of C. albicans cell growth.

Experiment 5: Coating of wettable polypropylene spunbond (SB) with 20 k PEG and 900 k PEO to test their ability to inhibit C. albicans cell adherence using the same SKINETHIC® reconstituted vaginal epithelium tissue model in Experiment 4.

In addition to PEG and PEO polymer solution samples, PEG treated nonwoven material, in this case wettable polypropylene spunbond (SB) fabric, was also tested and compared with untreated SB control. Samples of wettable nonwoven polypropylene SB fabric were coated separately with 20 k PEG and 900 k PEO. All showed significant results for the prevention of yeast cell adherence using SKINETHIC® reconstituted vaginal epithelium tissue.

The following materials and method were used:

PEG 20 k

PEO 900 k

1× sterile PSB buffer

13.3 gsm wettable polypropylene SB (from KOTEX® Tampon outliner)

5 mg/ml polymer solutions were prepared using sterilized 1×PBS solution, and then were coated onto SB through dipping followed by oven drying at 85° C. for 4 hours.

Prior to testing, each 0.5 cm² SKINETHIC® human epithelial tissue was moved into a 24-well plate with 0.5 ml fresh SKINETHIC® Maintenance (at room temperature). Then SB (polymer treated and untreated) were re-wetted by 1×PSB buffer before they were gently applied to and laid flat on top of the tissue cell surface using sterilized tweezers.

Carefully, 200 μl sterile 1×PSB (at room temperature) was added into each well, and incubated for 30 minutes at 37° C. Then the SB was lifted to half open by sterilized tweezers and 0.5 ml of the yeast TSB suspension (at a concentration of 1×10⁶ cfu/ml) was added into each well.

After about two hours of incubation, the liquid and SB samples were removed from wells. The wells were rinsed thoroughly three times with PBS to remove all non-bound yeast.

The tissue was cut with a sterilized scalpel and rinsed by PBS again. Then the bound bacteria were re-grown in 5 ml TSB and the whole system was incubated at 37° C. with shaking for 4 hours. The TSB was plated on Sabouraud's dextrose agar plates after 1×, 0.1×, and 0.01× serial dilutions. The plates were incubated overnight at 35° C. The number of colonies on each plate was counted and calculated back to the number of yeast attached to the cells in each well.

FIG. 8 shows the results of adherence testing for wettable SB coated with PBS control, untreated SB, SB coated with PEG and 900 k PEO. Again the bar in FIG. 8 represents mean±SEM and N=4/group.

Both 20 k PEG and 900 k PEO treated nonwoven wettable SB showed significant results in preventing yeast cell adherence using SKINETHIC® reconstituted vaginal epithelium tissue model. Thus, the feasibility of using PEG and PEO based hydrophilic polymers and PEO based amphiphilic block copolymers for urogenital infection inhibition was demonstrated.

Since the water soluble PEG and PEO coating can be gradually dissolved and released to vaginal canal environment during the tampon use, the released PEG and PEO not only can function as a moisturizer to ease vaginal dryness, they can lubricant the vaginal wall and reduce/prevent pathogenic microorganism's adhesion.

The PEO and PEG coating on wettable SB-tampon outliner was successfully achieved by dip-coating, also known as dip and squeeze. Alternative methods include spray coating, ink-jet printing and other techniques known in the art.

The composition may be applied to vaginal health products like, for example, moisturizer, gel, jelly, cream, insert (tablet), ointment, foam. The composition may also be applied to a feminine hygiene product, like, for example, a tampon, pad, or pant liners, which can reduce/prevent pathogenic microorganism's adhesion and prevent pathogen associated malodor.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1) A composition to inhibit the adherence of pathogenic microorganisms comprising a water soluble PEO polymerpolymer wherein the composition prevents adherence of said pathogenic microorganisms in a urogenital tract of a mammal.

2) The composition of claim 1 wherein said water soluble PEO polymer is present in a concentration of between 2 and 20 weight percent.

3) The composition of claim 1 wherein said water soluble PEO polymer is present in a concentration of between 5 and 10 weight percent.

4) The composition of claim 1, wherein said pathogenic microorganism is a fungal pathogen.

5) The composition of claim 1, wherein said pathogenic microorganism is a bacterial, viral or trichomonial pathogen.

6) The composition of claim 1, wherein the composition is applied to a vaginal health product selected from the group consisting of moisturizer, gel, jelly, cream, inserts, ointment and foam.

7) The composition of claim 1 wherein said composition is applied to a feminine hygiene product selected from the group consisting of tampons, feminine pads, feminine wipes or panty liners.

8) The composition of claim 1, wherein said water soluble PEO polymer is polyethylene glycol or polyethylene oxide with a chemical formula is —(OCH2CH2)n-O or H—(OCH2CH2)n-OH.

9) The composition of claim 1, wherein said water soluble PEO polymer is an amphiphilic block copolymer.

10) The composition of claim 9, wherein said amphiphilic block copolymers are PEO based block copolymers with a chemical formula of PEOm-Xp-PEOn, where PEO is —(OCH2CH2)n-O and X is PPO(—(OCH2CH2CH2)m-), PLGA (poly(lactic-co-glycolic acid), PMMA (polymethyl methacrylate), or PBO (polybutylene oxide) and m, n and p can be the same or different.

11) The composition of claim 9 in which said amphiphilic block copolymers have conformations selected from the group consisting of rod-like, star-like, back-bone and side chain-like.

12) A feminine hygiene product having an outer surface, said outer surface having thereon a composition to inhibit the adherence of pathogenic microorganisms to cells, comprising water soluble PEO.

13) The feminine hygiene product of claim 12 wherein said polyethylene glycol or polyethylene oxide polymer and/or block copolymers is present in a concentration of between 2 and 20 weight percent.

14) The feminine hygiene product of claim 12 wherein said copolymer is present in a concentration of between 5 and 10 weight percent.

15) The feminine hygiene product of claim 12, wherein said water soluble PEO polymer is polyethylene glycol or polyethylene oxide with a chemical formula is —(OCH2CH2)n-O or H—(OCH2CH2)n-OH.

16) The feminine hygiene product of claim 12, wherein said water soluble PEO polymer is an amphiphilic block copolymer.

17) The composition of claim 15, wherein said amphiphilic block copolymer has a chemical formula of PEOm-Xp-PEOn, where PEO is —(OCH2CH2)n-O and X is PPO(—(OCH2CH2CH2)m-), PLGA (poly(lactic-co-glycolic acid), PMMA (polymethyl methacrylate), or PBO (polybutylene oxide) and m, n and p can be the same or different.

18) A method of inhibiting adherence of pathogenic microorganisms to the vaginal wall including the steps of:
applying to a feminine hygiene product outer cover a composition comprising polyethylene glycol or polyethylene oxide polymer and/or block copolymers and;
properly using said product.

19) A vaginal insert product for preventing a urogenital infection in a mammal comprising an effective amount of soluble PEO polymers and copolymers therethereby inhibiting the adherence of pathogenic microorganisms to cells.

20) The vaginal insert product of claim 19 wherein said insert is a moisturizer.
